(19) United States
(12) Patent Application Publication
(10) Pub. No.: US 2025/0336549 A1
(43) Pub. Date: Oct. 30, 2025

(54) SYSTEM FOR SYMPTOM SIMILARITY MEASUREMENT USING LARGE LANGUAGE MODEL AND SELF-SUPERVISED LEARNING

(71) Applicant: 3billion, Inc., Seoul (KR)

(72) Inventors: Won Chan JEONG, Seoul (KR); Ho Heon KIM, Seoul (KR); Joo Yeup BAEK, Seoul (KR)

(73) Assignee: 3billion, Inc., Seoul (KR)

(21) Appl. No.: 19/176,905

(22) Filed: Apr. 11, 2025

(30) Foreign Application Priority Data
Apr. 24, 2024 (KR) 10-2024-0054665

(51) Int. Cl. G16H 70/60

(52) U.S. Cl. CPC G16H 70/60

(57) ABSTRACT

A symptom similarity measurement system using a large language model and self-supervised learning includes: a symptom embedding generation unit converting symptom text information of disease symptoms into embedding values for each symptom and generating disease symptom data using a large language model; a patient data generation unit generating synthetic data, a set of symptoms of a specific disease formed by randomly sampling all symptoms known for the specific disease; and a symptom model training unit performing self-supervised learning on a symptom model using the synthetic data.

### CROSS-REFERENCE TO RELATED APPLICATIONS

This application claims the benefit of Korean Patent Application No. 10-2024-0054665, filed on Apr. 24, 2024, the entire contents of which are incorporated herein by reference.

### BACKGROUND

### Technical Field

The present disclosure relates to a symptom similarity measurement system using a large language model and self-supervised learning, and more specifically, to a system measuring symptom similarity between a patient's symptoms and disease symptom data using a large language model.

### Description of Related Art

For accurate diagnosis of a patient, it is important to suggest potential diseases based on observed symptoms of the patient. Since multiple diseases may not share the same symptoms or the symptoms of a specific disease may not always appear with the same frequency, it may be difficult to compare disease symptoms with ‘patients’ symptoms to determine the correct disease.

Recently, symptom information associated with specific diseases has been systematically compiled, and methods to aid diagnosis by comparing a patient's symptoms with known symptoms of diseases have been proposed.

There are provided methods for measuring the similarity between a patient's symptom set and a disease symptom set based on shared symptoms between the observed symptoms of the patient and the known symptoms of each disease or for measuring the similarity between the patient's symptom set and the disease symptom set based on the similarity of protein interaction networks associated with each symptom.

The calculation of symptom similarity between the patient's symptoms and the disease symptoms becomes an important indicator in narrowing potential disease candidates and guiding clinicians toward an accurate diagnosis.

Conventional symptom similarity measurement methods are based on the human phenotype ontology (HPO) structure. The HPO provides standardized terms for describing symptoms, and has a directed acyclic graph (DAG) structure that defines subordinative relationships among symptom terms. More detailed symptoms are positioned deeper in the hierarchy of the HPO structure.

The conventional symptom similarity measurement methods represent a patient's symptoms and disease symptoms as concepts within the HPO and calculate symptom similarity by comparing the structural characteristics of the HPO, such as distance, depth, and so on.

However, conventional symptom similarity measurement methods that rely on the HPO structure have several problems. First, conventional such symptom similarity measurement methods often do not reflect linguistic and medical contexts of actual symptoms, due to recognizing symptoms as objects in a graph. For instance, derivative symptoms may be medically similar but may appear far apart in a graph. Second, such conventional symptom similarity measurement methods can weight disease-specific symptoms based on the depth of the HPO hierarchy, but may reduce the discriminative power in diseases that share similar symptoms.

Specifically, in case of rare diseases, since there are many diseases sharing similar symptoms due to the complexity of symptoms, it is difficult to correctly calculate symptom similarity. Therefore, there is a need for a symptom similarity measurement technique that overcomes the problems.

### PATENT LITERATURE

### Patent Documents

Patent Document 1: KR Patent No. 10-2167697 (Granted on Oct. 13, 2020)

### DISCLOSURE

### Technical Problem

The present disclosure has been made to solve the above-mentioned problems occurring in the prior art, and in an aspect of the present disclosure, an object of the present disclosure is to provide a symptom similarity measurement system capable of distinguishing diseases sharing similar symptoms.

### Technical Solution

To accomplish the above-mentioned objects, according to an aspect of the present invention, there is provided a symptom similarity measurement system using a large language model and self-supervised learning including: a symptom embedding generation unit converting symptom text information of disease symptoms into embedding values for each symptom and generating disease symptom data using a large language model; a patient data generation unit generating synthetic data, a set of symptoms of a specific disease formed by randomly sampling all symptoms known for the specific disease; and a symptom model training unit performing self-supervised learning on a symptom model using the synthetic data.

The symptom text information is collected from the human phenotype ontology (HPO).

The synthetic data is a set of the disease symptom data.

The patient data generation unit forms a set of symptoms for a specific disease by randomly sampling a complete set of symptoms known as symptoms of the specific disease, forms a set of noise symptoms unrelated to the specific disease by sampling symptoms of diseases besides the specific disease, and then, generates synthetic data by uniting the set of symptoms for the specific disease and the set of noise symptoms.

The number of noise symptoms in the set of noise symptoms is equal to or less than the number of symptoms of the specific disease in the set of symptoms for the specific disease.

The symptom model training unit performs self-supervised learning using contrastive learning.

The symptom model training unit, to perform the contrastive learning, uses one of simple framework for contrastive learning f representations (SimCLR), simple contrastive learning of sentence embeddings (SimCSE), contrastive unsupervised representations for reinforcement learning (CURL), momentum contrast for unsupervised visual representation learning (MoCo), Barlow Twins, and bootstrap your own latent (BYOL).

The symptom similarity measurement system further includes a symptom similarity calculation unit, calculating symptom similarity between a patient's symptoms and disease symptoms, between a patient's symptoms and genetic variation symptoms, or between different patients' symptoms using the symptom model.

The symptom embedding generation unit generates patient symptom data by converting a patient's symptom information into embedding values on patient symptoms, and also generates genetic variation symptom data by converting genetic variation symptom information into embedding values on genetic variation symptoms.

The symptom similarity calculation unit calculates symptom similarity using one or more of cosine similarity, Jaccard similarity, and Euclidean distance.

### Advantageous Effects

According to the present invention, the following effects can be achieved.

The present invention can convert symptom text information of disease symptoms into embedding values that reflect linguistic and medical contexts.

The present invention can effectively calculate symptom similarity through self-supervised learning, which enables clear distinction between diseases with similar symptoms.

The present invention can identify genes closely related to the patient's symptoms or other patients with similar symptoms through the symptom similarity calculation.

The present invention can calculate symptom similarity with higher accuracy as more symptom data is accumulated.

In addition, other features and advantages of the present invention may be clearly understood through embodiments of the present invention.

### BRIEF DESCRIPTION OF THE DRAWINGS

FIG. 1 is a diagram illustrating the configuration of a symptom similarity measurement system according to an embodiment of the present invention.

FIG. 2 is a diagram illustrating the process in which a symptom embedding generation unit converts symptom text information into embedding values according to an embodiment of the present invention.

FIG. 3 is a diagram illustrating the process in which a patient data generation unit generates synthetic data according to an embodiment of the present invention.

FIG. 4 is a diagram illustrating the process in which the patient data generation unit generates synthetic data with noise symptoms according to an embodiment of the present invention.

FIG. 5 is a diagram illustrating the process in which a symptom model training unit learns the synthetic data according to an embodiment of the present invention.

FIG. 6 is a diagram illustrating the process in which a symptom similarity calculation unit calculates a patient's symptom similarity according to an embodiment of the present invention.

### DETAILED DESCRIPTION

In this specification, reference numbers are assigned to components in the drawings, and the same reference numbers are used for identical components even if the components appear in different drawings. It should be noted that singular expressions in the specification include plural meanings unless specifically stated otherwise. It should be also understood that the terms “comprises” and/or “comprising” in the specification do not exclude the presence or addition of one or more other components besides components described in the specification.

The objects, features, and advantageous of the present invention will be described in detail through the following preferable exemplary embodiments with reference to the accompany drawings. As those skilled in the art would realize, the described embodiments may be modified in various different ways, all without departing from the spirit or scope of the present invention. On the contrary, exemplary embodiments introduced herein are provided to make disclosed contents thorough and complete and to sufficiently transfer the spirit of the present invention to those skilled in the art.

Hereinafter, preferred embodiments of the present invention, designed to overcome the aforementioned problems, will be described in detail with reference to the accompanying drawings.

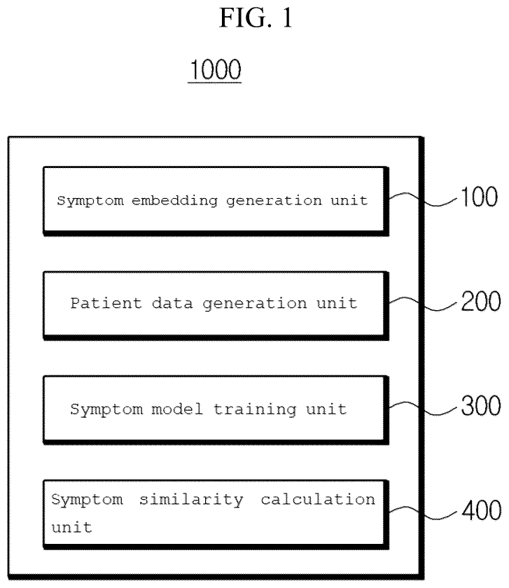

FIG. 1 is a diagram illustrating the configuration of a symptom similarity measurement system according to an embodiment of the present invention.

Referring to FIG. 1, a symptom similarity measurement system 1000 according to an embodiment of the present invention may include a symptom embedding generation unit 100, a patient data generation unit 200, a symptom model training unit 300, and a symptom similarity calculation unit 400.

The symptom embedding generation unit 100 may convert symptom text information of disease symptoms into embedding values for each symptom and generate disease symptom data using a large language model (LLM).

The patient data generation unit 200 may generate synthetic data, which is a set of symptoms of a specific disease formed by randomly sampling all symptoms known for the specific disease.

The symptom model training unit 300 may perform self-supervised learning on a symptom model using the synthetic data.

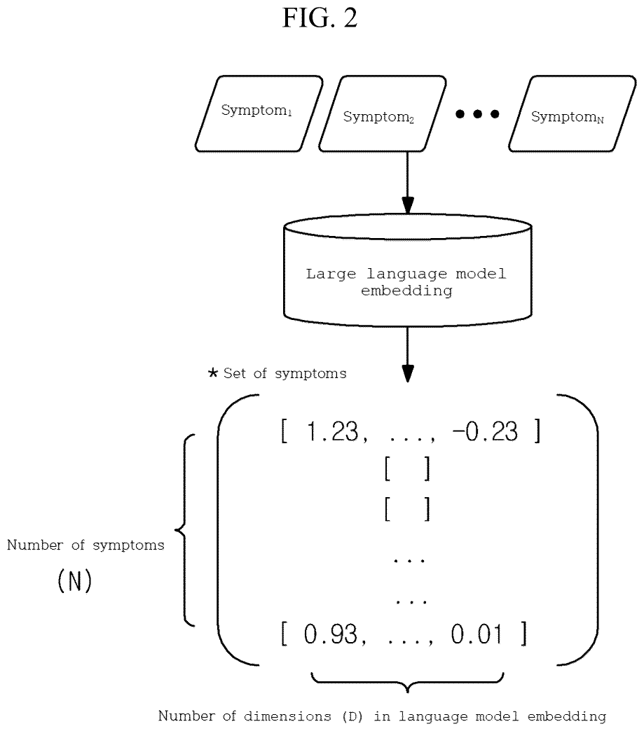

FIG. 2 is a diagram illustrating the process in which a symptom embedding generation unit converts symptom text information into embedding values according to an embodiment of the present invention.

Referring to FIG. 2, the symptom embedding generation unit 100 may convert symptom texts into embedding values by using a large language model (LLM) to express symptom text information numerically.

The large language model (LLM) is a large neural network model used in natural language processing (NLP). The large language model (LLM) is pre-trained through large-scale datasets to acquire general language understanding capabilities. The pre-trained large language model can be fine-tuned to be suitable for specific tasks. Therefore, according to an embodiment of the present invention, the large language model (LLM) can perform symptom text embedding and can be fine-tuned to calculate symptom similarity.

The large language model (LLM) can use a model opened publicly. For instance, the large language model (LLM) can use one of Mistral models opened in OpenAI's GPT series, Google's BERT, or Mistral AI.

According to an embodiment of the present invention, when N symptoms (Symptom 1, . . . , Symptom N) are input, each symptom text information is converted into a vector, which is the number of dimensions (D) in language model embedding. The vector is a D-dimensional vector. Additionally, the corresponding vectors, equal to the number (N) of symptoms, are created into a two-dimensional (N×D) matrix.

The symptom embedding generation unit 100 may generate patient symptom data by converting a patient's symptom information into embedding values on patient symptoms, and also generate genetic variation symptom data by converting genetic variation symptom information into embedding values on genetic variation symptoms.

The patient symptom information may include information related to symptom phenotypes. The symptom phenotypes refer to observable characteristics or symptoms that appear in an individual due to a specific disease. For example, Moyamoya disease may present symptoms such as renovascular hypertension, anterior cerebral artery stenosis, cerebellar agenesis, and aortic stenosis. In other words, the symptom phenotype of Moyamoya disease may include renovascular hypertension, anterior cerebral artery stenosis, cerebellar agenesis, and aortic stenosis.

Genetic variation refers to the occurrence of different forms of genes within a population. In the present invention, genetic variation may refer to a phenomenon in which the DNA base sequence in specific gene changes. Genetic variation can change gene functions, as a result, may cause diseases.

Accordingly, the symptom embedding generation unit 100 can generate symptom data for the genetic variation by converting symptom information caused by genetic variations into embedding values of symptoms related to the genetic variation, thus identifying genetic variations associated with symptoms.

The symptom text information may be collected from the human phenotype ontology (HPO).

The human phenotype ontology (HPO) is a database that provides standardized vocabulary related to human phenotypes. The human phenotype ontology (HPO) comprehensively categorizes and describes a wide range of phenotypes, including symptoms and clinical characteristics of diseases.

The symptom text information may include symptom descriptions and comments as specified in the HPO. The symptom phenotype may be a standardized symptom described in the HPO.

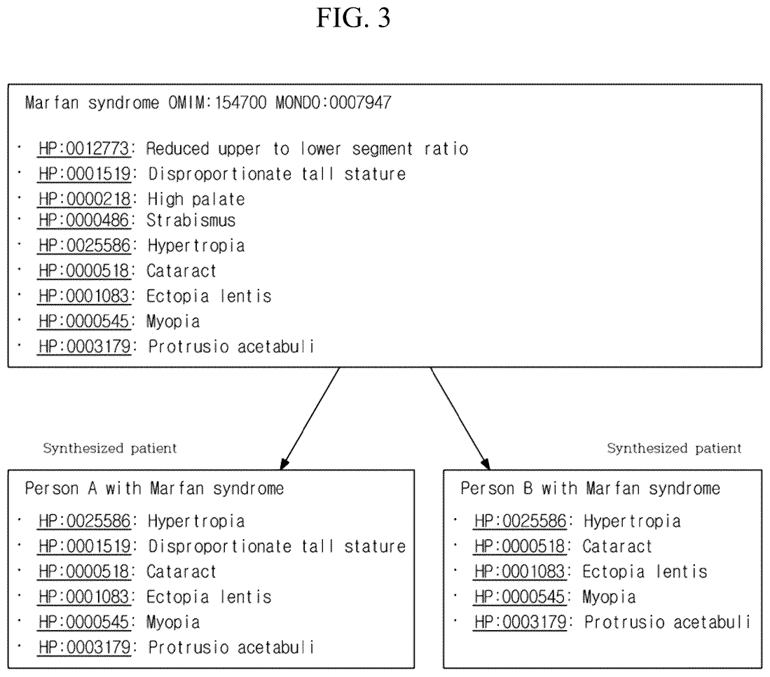

FIG. 3 is a diagram illustrating the process in which the patient data generation unit generates synthetic data according to an embodiment of the present invention.

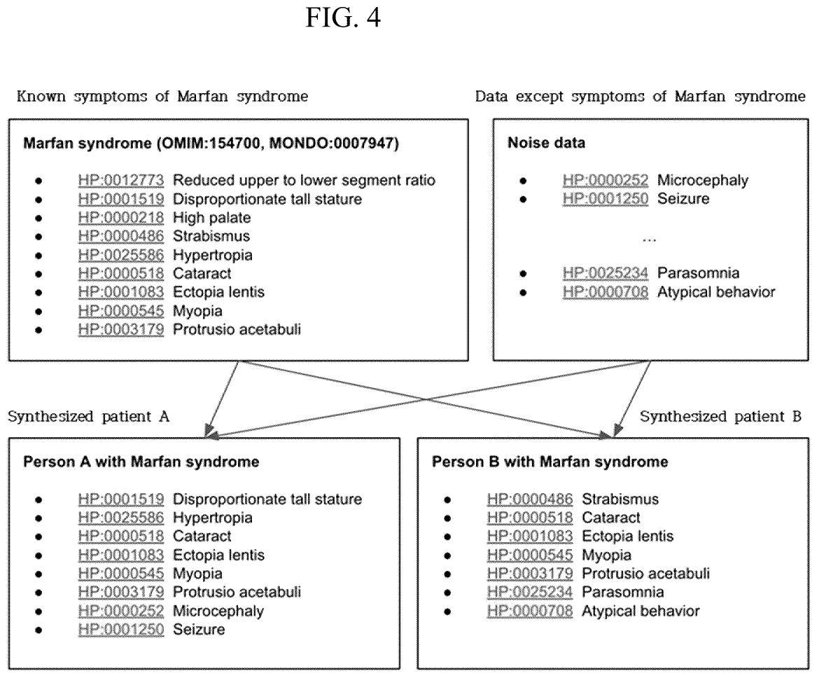

FIG. 4 is a diagram illustrating the process in which the patient data generation unit generates synthetic data with noise symptoms according to an embodiment of the present invention.

Referring to FIGS. 3 and 4, the patient data generation unit 200 of the present invention may generate synthetic data as training data for the symptom model.

The symptom similarity measurement system 1000 of the present invention utilizes self-supervised learning to train the symptom model for calculating symptom similarity.

Self-supervised learning (SSL) is a technique in which a model learns patterns to find rules in data and obtain better representation capabilities using unlabeled data. In the self-supervised learning (SSL), the learning algorithm automatically generates labeled data from unlabeled data and uses the labeled data for training.

Using actual patient data to train a symptom model has many difficulties. The actual patient data is considered a patient's personal information, requiring careful handling. Furthermore, the actual patient data is limited in data quantity and cannot show all possible symptom combinations for a specific disease.

Specifically, in case of rare diseases, it is more difficult to collect sufficient patient data.

Here, rare diseases are defined as diseases that affect only a very small portion of the population. In the United States, diseases affecting fewer than 1 in 5,000 people are classified as rare diseases, whereas in Japan, diseases affecting fewer than 1 in 2,500 people are classified as rare diseases.

Additionally, many rare diseases are caused by genetic variations, and it is difficult to obtain data on each symptom induced by a specific genetic variation.

Accordingly, the symptom similarity measurement system 1000 of the present invention may generate synthetic data for training the symptom model.

According to an embodiment of the present invention, the synthetic data may be a set of disease symptom data, where the symptom embedding generation unit 100 converts the symptom text information into embedding values.

The patient data generation unit 200 may generate synthetic data by randomly sampling a portion of disease symptom data for a specific disease. That is, synthetic data for a patient with disease A is generated by sampling K disease symptoms from the complete set of disease symptoms associated with the disease A.

In another embodiment, the patient data generation unit 200 may form a set of symptoms for a specific disease by randomly sampling a complete set of symptoms known as symptoms of the specific disease, form a set of noise symptoms unrelated to the specific disease by sampling symptoms of diseases besides the specific disease, and then, generate synthetic data by uniting the set of symptoms for the specific disease and the set of noise symptoms.

The noise symptoms refer to symptoms, which are unrelated to or not directly caused by a patient's disease, among various symptoms observed in the patient.

The number of noise symptoms in the set of noise symptoms may be equal to or less than the number of symptoms of the specific disease in the set of symptoms for the specific disease.

Specifically, let P be a set of symptoms for all diseases, and let D be a set of symptoms associated with disease A, wherein D is a subset of P. The patient data generation unit 200 may sample K disease symptoms from the set of symptoms for disease A to create a synthetic symptom set S_S. Additionally, the patient data generation unit 200 may unite a set S_N, in which J symptoms except the set D of symptoms for the disease A are sampled, (S_N⊆P-D) and the synthetic symptom set S_S to generate synthetic data with added noise.

FIG. 3 lists known symptoms of Marfan syndrome. Each symptom is embedded into disease symptom data by the symptom embedding generation unit 100. The patient data generation unit 200 may sample a portion of the disease symptom data corresponding to each symptom to generate synthetic data for patients A and B with Marfan syndrome.

The patient data generation unit 200 may generate synthetic data that includes both symptoms for a specific disease and noise symptoms.

For example, microcephaly, seizures, parasomnia, and atypical behavior are noise symptoms, which are not symptoms of Marfan syndrome. The patient data generation unit 200 may form a set of noise symptoms by sampling microcephaly and seizures among the noise symptoms and form another set of noise symptoms by sampling parasomnia and atypical behavior. The patient data generation unit 200 may unite the set of noise symptoms with the set of symptoms of Marfan syndrome to generate synthetic data.

In the synthetic data containing noise symptoms, the number of noise symptoms should be equal to or less than the number of symptoms associated with the specific disease. For example, if the number of symptoms of Marfan syndrome in the synthetic data A is six, the number of noise symptoms should not exceed six.

According to an embodiment of the present invention, the patient data generation unit 200 may generate synthetic data not only for patients with a single disease but also for patients with multiple diseases. For example, the patient data generation unit 200 may combine disease symptom data for Marfan syndrome and disease symptom data for Moyamoya disease to generate synthetic data representing a patient who has both Marfan syndrome and Moyamoya disease.

According to another embodiment of the present invention, the patient data generation unit 200 may generate synthetic data by adding weight to specific symptom data among disease symptom data. For example, the patient data generation unit 200 may assign a higher weight to nearsightedness symptom data and a lower weight to cataract symptom data in disease symptom data of Marfan syndrome, to generate synthetic data representing a patient with severe myopia but mild cataracts.

Additionally, the patient data generation unit 200 may assign a higher weight to disease symptom data of one disease among multiple diseases but a lower weight to disease symptom data of other diseases. According to the embodiment of the present invention, the patient data generation unit 200 can generate synthetic data representing a patient with multiple diseases but with predominant symptoms of only one disease.

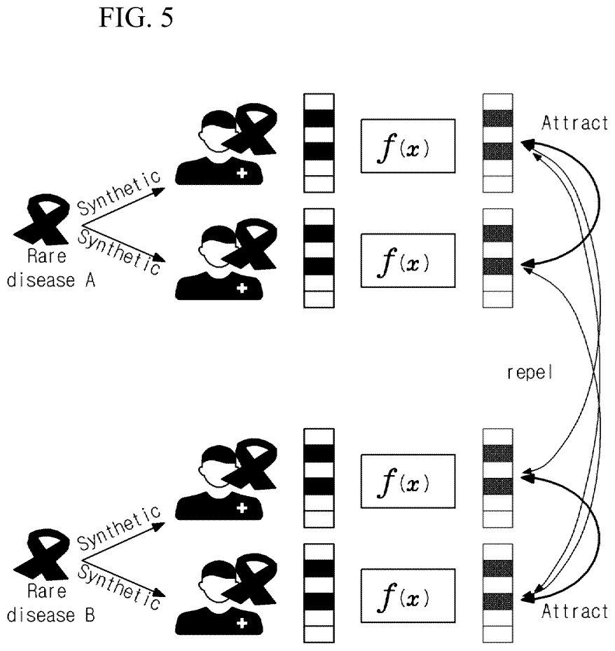

FIG. 5 is a diagram illustrating the process in which a symptom model training unit learns the synthetic data according to an embodiment of the present invention.

Referring to FIG. 5, the symptom model training unit 300 may perform self-supervised learning using contrastive learning.

According to an embodiment of the present invention, to train differentiation between various diseases, a discriminative approach within the self-supervised learning may be selected.

The discriminative approach focuses on classification problems, emphasizing differences between various classes or categories, so that the learning model can distinguish the differences between various classes or categories. The discriminative approach defines clear boundaries between data points, focusing on classification of each data point.

According to an embodiment of the present invention, the symptom model training unit 300 may use contrastive learning in the discriminative approach.

The contrastive learning trains the model to bring representations of data pairs with the same origin closer while putting representations of different data pairs distance.

According to an embodiment of the present invention, the symptom model training unit 300 may generate synthetic data with K and K′ symptoms each by sampling a rare disease A with the patient data generation unit 200. The symptom model training unit 300 may define an embedding function based on the received synthetic data. The symptom model training unit 300 represents that similar embedding functions are positioned closer (attract) and different embedding functions are positioned farther apart (repel) in the embedding space. Therefore, the embedding functions made through sampling of the rare disease A are positioned closely in the embedding space.

Furthermore, if the symptom model training unit 300 generates synthetic data with K and K′ symptoms each by sampling a rare disease B and defines an embedding function, the embedding function made through sampling of the rare disease A and the embedding function made through sampling of the rare disease B are positioned far apart in the embedding space.

The symptom model finally trained by the symptom model training unit 300 aggregates and returns N symptom phenotypes of the rare disease A into a single vector. The vector represents the rare disease A and serves as a reference for determining whether symptoms inputted in the symptom similarity measurement system of the present invention correspond to the rare disease A or not.

To perform the contrastive learning, the symptom model training unit 300 may utilize one of simple framework for contrastive learning of representations (SimCLR), simple contrastive learning of sentence embeddings (SimCSE), contrastive unsupervised representations for reinforcement learning (CURL), momentum contrast for unsupervised visual representation learning (MoCo), Barlow Twins, and bootstrap your own latent (BYOL).

SimCLR, which is one of frameworks for contrastive learning, extracts feature vectors from converted embedding values and maps the extracted feature vectors into lower-dimensional final vectors. SimCLR expresses that similar data points are positioned closer while different data points are positioned farther apart.

SimCSE, which is one of frameworks for contrastive learning, converts a single data point into multiple slightly varied embedding values. SimCSE expresses that different embeddings converted from the same data point are positioned closer, while embeddings converted from different data points are positioned farther apart.

CURL, which is one of frameworks for contrastive learning, applies multiple transformations to data points and extracts features. CURL performs contrastive learning of the extracted features, and expresses that similar states are positioned closer and different states are positioned farther apart.

MoCo is one of the contrastive learning techniques. MoCo processes each data as a Query and a Key, and arranges Queries and Keys derived from the same data closer, while Queries and Keys derived from different data farther apart.

Barlow Twins (Self-supervised learning via redundancy reduction) is one of unsupervised learning methods. Barlow Twins extract independent and informative features from two variations of the same input data. Barlow Twins generates two different ‘views’ of the data, and applies the same neural network to both two different ‘views’ to obtain two outputs. Thereafter, Barlow Twins maximizes similarity of the two outputs but minimizes redundancy of each feature.

Bootstrap your own latent (BYOL) is one of unsupervised learning methods using deep neural networks. BYOL uses two networks: an ‘online network’ and a ‘target network.’ The two networks learn to extract features from two differently augmented versions of the same input data. BYOL makes the two networks use each other as learning objectives such that each network predicts the output of the other.

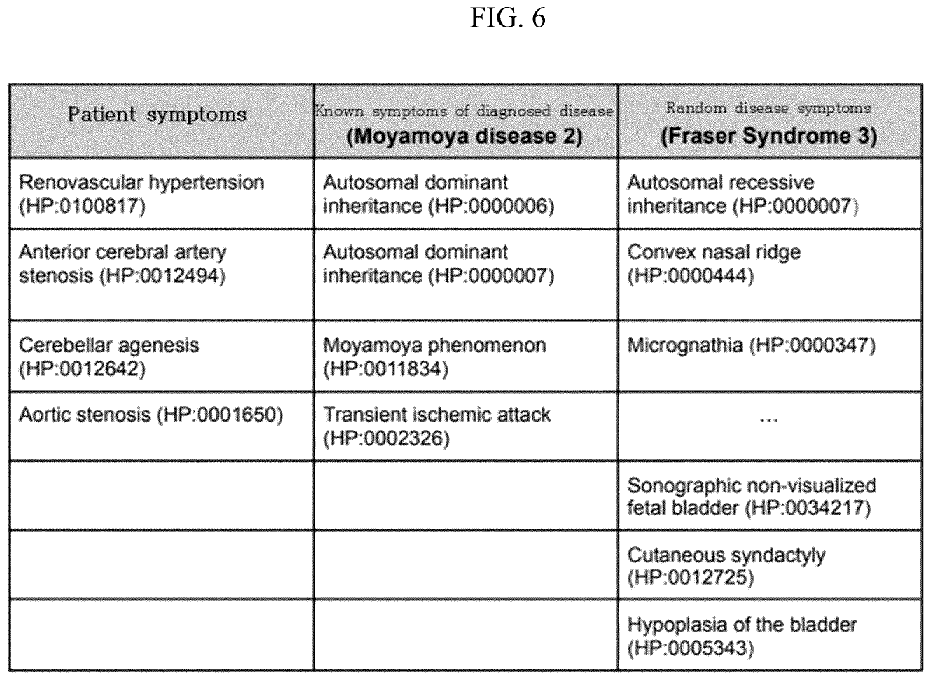

FIG. 6 is a diagram illustrating the process in which a symptom similarity calculation unit calculates a patient's symptom similarity according to an embodiment of the present invention.

Referring to FIG. 6, the symptom similarity calculation unit 400 may calculate symptom similarity using the symptom phenotype vectors generated by the symptom embedding generation unit 100.

The symptom similarity calculation unit 400 may calculate symptom similarity between a patient's symptoms and disease symptoms, between a patient's symptoms and genetic variation symptoms, or between different patients' symptoms using the symptom model.

The symptom similarity calculation unit 400 may receive a patient's disease symptom data and calculate the symptom similarity between the received patient's disease symptom data and the learned disease symptom data using the symptom model.

Additionally, the symptom similarity calculation unit 400 may calculate the symptom similarity between disease symptom data caused by genetic variations, among the received patient's disease symptom data and the disease symptom data.

The symptom similarity calculation unit 400 may receive disease symptom data of two or more patients and calculate the symptom similarity between the disease symptom data of one patient and the disease symptom data of the other.

According to an embodiment of the present invention, the symptom similarity calculation unit 400 may provide information on whether the received patient symptoms correspond to symptoms of a particular disease, which genetic mutation may have caused the received patient symptoms, or who has the same disease.

The symptom similarity calculation unit 400 may calculate symptom similarity using one or more of cosine similarity, Jaccard similarity, and Euclidean distance.

Cosine similarity is a method to calculate using a cosine angle between two vectors. The method focuses on vector orientation rather than vector magnitude, assigning a high similarity score when vectors have the same orientation and a low score when vectors have different orientations.

Jaccard similarity is a method to measure similarity between two sets, and uses a value that divides the size of intersection of the two sets by the size of union of the two sets.

Euclidean distance is a method to measure the straight distance between two points.

According to an embodiment of the present invention, an actual patient may exhibit symptoms such as renovascular hypertension, anterior cerebral artery stenosis, cerebellar agenesis, and aortic stenosis. The symptom embedding generation unit 100 of the present invention may convert symptom phenotypes into embedding values as shown in [Equation 1].

$$[ Equation 1 ]$$

$${v}_{patient} = [ - 0.01224341 , 0.02224341 , … , - 0.03224341 , 0.048243 ] \\ {v}_{moyamoya disease} = [ - 0.01448052 , 0.01769544 , … , - 0.01872689 , - 0.02514333 ] \\ {v}_{fraser Syndrome} = [ - 0.02173538 , 0.01032189 , … , 0.00790377 , - 0.01066733 ]$$

If the symptom model of the present invention has been trained on the symptoms of Moyamoya disease and Fraser Syndrome by the symptom model training unit 300, the symptom similarity calculation unit 400 may calculate the symptom similarity between the patient's symptoms and disease symptoms as shown in [Equation 2].

$$\begin{matrix}{v}_{parient} @ {v}_{moyamoya disease} = 0.7595 \\ {v}_{patient} @ {v}_{fraser Syndrome} = - 0.1474\end{matrix} , \qquad [ Equation 2 ]$$

wherein @ represents cosine similarity between vectors.

The symptom similarity calculation unit 400 may determine that the patient's symptoms have a higher similarity to Moyamoya disease than to Fraser Syndrome.

While the embodiments have been described with reference to limited embodiments and figures, those skilled in the art will recognize that various modifications and alterations can be made based on the above disclosure. For example, the described techniques may be executed in a different order, and/or components of the described structures and devices may be combined or coupled with forms different from the described methods, or may be replaced, or substituted with different elements or equivalents while still achieving appropriate results.

Therefore, other implementations, embodiments, and equivalents that fall within the scope of the following claims are also included in the scope of the present invention.

1. A symptom similarity measurement system using a large language model and self-supervised learning, comprising:
a symptom embedding generation unit converting symptom text information of disease symptoms into embedding values for each symptom and generating disease symptom data using a large language model;
a patient data generation unit generating synthetic data, a set of symptoms of a specific disease formed by randomly sampling all symptoms known for the specific disease; and
a symptom model training unit performing self-supervised learning on a symptom model using the synthetic data.

2. The symptom similarity measurement system according to claim 1, wherein the symptom text information is collected from the human phenotype ontology (HPO).

3. The symptom similarity measurement system according to claim 2, wherein the synthetic data is a set of the disease symptom data.

4. The symptom similarity measurement system according to claim 3, wherein the patient data generation unit forms a set of symptoms for a specific disease by randomly sampling a complete set of symptoms known as symptoms of the specific disease, forms a set of noise symptoms unrelated to the specific disease by sampling symptoms of diseases besides the specific disease, and then, generates synthetic data by uniting the set of symptoms for the specific disease and the set of noise symptoms.

5. The symptom similarity measurement system according to claim 4, wherein the number of noise symptoms in the set of noise symptoms is equal to or less than the number of symptoms of the specific disease in the set of symptoms for the specific disease.

6. The symptom similarity measurement system according to claim 3, wherein the symptom model training unit performs self-supervised learning using contrastive learning.

7. The symptom similarity measurement system according to claim 6, wherein the symptom model training unit, to perform the contrastive learning, uses one of simple framework for contrastive learning of representations (SimCLR), simple contrastive learning of sentence embeddings (SimCSE), contrastive unsupervised representations for reinforcement learning (CURL), momentum contrast for unsupervised visual representation learning (MoCo), Barlow Twins, and bootstrap your own latent (BYOL).

8. The symptom similarity measurement system according to claim 1, further comprising:
a symptom similarity calculation unit, calculating symptom similarity between a patient's symptoms and disease symptoms, between a patient's symptoms and genetic variation symptoms, or between different patients' symptoms using the symptom model.

9. The symptom similarity measurement system according to claim 8, wherein the symptom embedding generation unit generates patient symptom data by converting a patient's symptom information into embedding values on patient symptoms, and also generates genetic variation symptom data by converting genetic variation symptom information into embedding values on genetic variation symptoms.

10. The symptom similarity measurement system according to claim 9, wherein the symptom similarity calculation unit calculates symptom similarity using one or more of cosine similarity, Jaccard similarity, and Euclidean distance.
